(19) United States
(12) Patent Application Publication
(10) Pub. No.: US 2016/0038017 A1
(43) Pub. Date: Feb. 11, 2016

(54) RETRACTOR VISION SYSTEM

(71) Applicant: James C. ROBINSON, Atlanta, GA (US)

(72) Inventors: James C Robinson, Atlanta, GA (US); John Douglas Craft, Atlanta, GA (US); Miles Hunter Marks, Atlanta, GA (US)

(21) Appl. No.: 14/777,486

(22) PCT Filed: Mar. 17, 2014
(86) PCT No.: PCT/US14/30375

§ 371 Date: Sep. 15, 2015

Related U.S. Application Data
(60) Provisional application No. 61/786,595, filed on Mar. 15, 2013.

(51) Int. Cl. A61B 1/317; A61B 1/00; A61B 17/02; A61B 1/04; A61B 1/015; A61B 1/018; A61B 1/002

(52) U.S. Cl. CPC A61B 1/317; A61B 1/018; A61B 1/00154; A61B 1/002; A61B 1/04; A61B 1/0016; A61B 1/015; A61B 17/0218

(57) ABSTRACT

A retractor vision system having an elongate body with at least one longitudinal pathway defined therein is provided. The retractor vision system also has a vision module configured to fit at least partially in the longitudinal pathway. The vision module enables a surgeon to externally see an interior viewpoint of a patient during surgery.

### CROSS-REFERENCE TO RELATED APPLICATIONS

This application claims the benefit of priority to U.S. Provisional Application No. 61/786,595, filed Mar. 15, 2013, which is incorporated in its entirety by this reference.

### FIELD OF THE INVENTION

The present invention relates generally to a retractor for use in spinal and other surgical procedures. More specifically, the invention pertains to a retractor system having an integral vision component.

### BACKGROUND OF THE INVENTION

As can be appreciated, a surgeon's visual field is very important in surgery, such as, but not limited to spinal or skull base surgery. It becomes an issue in minimally invasive procedures because the incisions are necessarily smaller. One of the traditional solutions is to use an endoscope to enhance vision. An endoscope is a medical device consisting of a long, thin, flexible (or rigid) tube which has a light and a video camera. Images of the inside of the patient's body can be seen on a screen.

Another major advancement that has allowed surgeons to perform minimally invasive spine surgery is the use of retractors. As the incisions have shrunk over the years, retractors have been developed to hold back the tissue between the skin and the spine to see the area on which the surgeon is working; a tube has worked well.

Accordingly, it remains desirable in the pertinent art to provide the surgeon access to the working area and a field of vision large enough to perform the surgery.

### SUMMARY

Presented herein is a retractor vision system, comprising an elongate body having at least one longitudinal pathway defined therein. The retractor vision system also comprises a vision module configured to fit at least partially in the longitudinal pathway.

In one exemplified aspect, the elongate body is tubular with a substantially circular cross-sectional shape. In another aspect, the tubular design defines a working channel therein substantially surrounded by at least one longitudinal wall. The working channel can be dimensioned to accommodate the retractor size as well as the instruments that are required to work within the channel.

The vision module enables the surgeon to externally see an interior viewpoint during surgery. The vision system can be selectively moved in order to position the system to view under higher magnification, or to change the view to the region of interest. The vision module can comprise at least one optical transmissive element positioned therein the longitudinal pathway from substantially the proximal end of the elongate body to substantially the distal end of the elongate body. The optical transmissive element can comprise at least one of: a glass rod lens, a camera, a nano-camera, and other visualizing technology positioned therein the longitudinal pathway. A camera feed can be delivered to a screen for the surgeon to view the surgical area. Optionally, an eyepiece can be positioned at or near a proximal end of the optical transmissive element for the surgeon to view the surgical area.

Related methods of operation are also provided. Other apparatuses, methods, systems, features, and advantages of the retractor vision system will be or become apparent to one with skill in the art upon examination of the following figures and detailed description. It is intended that all such additional apparatuses, methods, systems, features, and advantages be included within this description, be within the scope of the retractor vision system, and be protected by the accompanying claims.

### DESCRIPTION OF THE FIGURES

FIG. 1 is a cut away front elevational view of a retractor vision system as used in spinal surgery;

FIG. 2 is a cut away perspective view of the retractor vision system of FIG. 1, showing a vision module having two optical transmissive elements;

FIG. 3. is a top plan view of an elongate body for a retractor vision system, showing a plurality of longitudinal pathways positioned therein the longitudinal wall of the elongate body;

FIG. 4 is a top plan view of an elongate body for a retractor vision system, showing a plurality of longitudinal pathways positioned therein the longitudinal wall of the elongate body where the longitudinal pathway is positioned on the inside portion of the longitudinal wall;

FIG. 5 is a top plan view of an elongate body for a retractor vision system, showing a plurality of longitudinal pathways positioned therein the longitudinal wall of the elongate body where the longitudinal pathway is positioned on the outside portion of the longitudinal wall;

FIG. 6 is a cut away front elevational view of a retractor vision system having a vision module comprising a pair of small cameras or nano-cameras

FIG. 7 is a partially exploded cut away perspective view of a retractor vision system having a worm gear for lowering and raising the vision module;

FIG. 8 is a perspective view of an elongate body for a retractor vision system;

FIG. 9 is a perspective view of an elongate body for a retractor vision system where the elongate body is substantially planar;

FIG. 10 is a perspective view of a retractor vision system having an elongate body and an outer retractor tube; and

FIG. 11 is a front elevational cut away view of a retractor vision system having an irrigation system.

### DESCRIPTION OF THE INVENTION

The present invention can be understood more readily by reference to the following detailed description, examples, and claims, and their previous and following description. Before the present system, devices, and/or methods are disclosed and described, it is to be understood that this invention is not limited to the specific systems, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. Those skilled in the relevant art will recognize that many changes can be made to the aspects described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “camera” includes aspects having two or more cameras unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

Terms used herein, such as “exemplary” or “exemplified,” are not meant to show preference, but rather to explain that the aspect discussed thereafter is merely one example of the aspect presented.

Additionally, as used herein, relative terms, such as “substantially”, “generally”, “approximately”, and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

In one aspect, presented herein is a retractor vision system 10 comprising an elongate body 100 defining at least one longitudinal pathway 110 and a separate working channel 130 therein. The working channel can be configured to provide access for at least one surgical instrument 125 to a surgical site on a patient. The retractor vision system 10 also comprises a vision module 200 configured to fit at least partially in the longitudinal pathway of the elongate body. The elongate body can be sized and shaped to engage clamps 124, retractors and/or other surgical devices to maintain the elongate body 100 in a desired position relative to the patient.

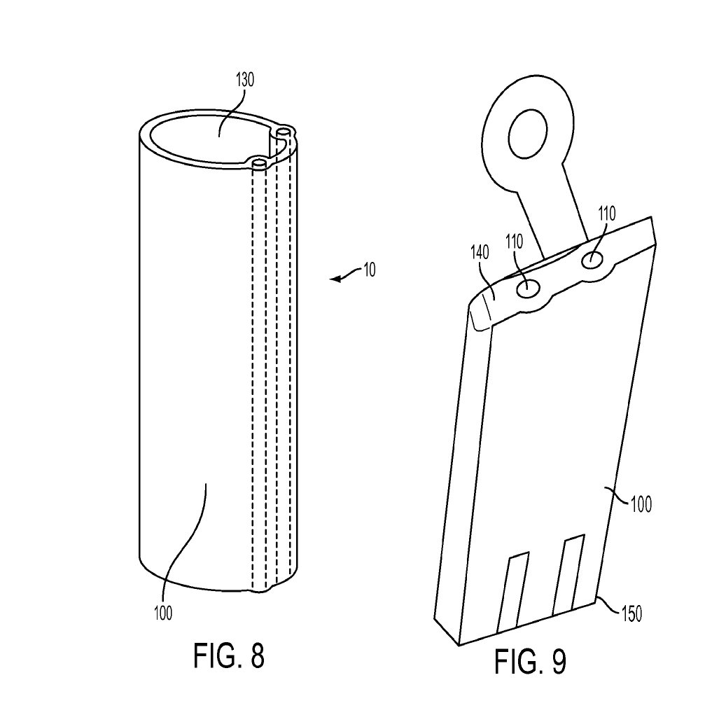

In one exemplified aspect, the elongate body 100 can be tubular in shape having at least one longitudinal wall 120 that defines at least a portion of the working channel 130, as illustrated in FIG. 8. In another aspect, the elongate body can be substantially circular in cross-sectional shape. However, it is contemplated that the elongate body can have various cross-sectional shapes. For example and not meant to be limiting, the elongate body can have cross-sectional shapes that are substantially oval, square, rectangular, hexagonal, octagonal, and the like. In one aspect, the longitudinal wall of the elongate body can be entirely closed such that the working channel 130 is closed. Optionally, however, at least one aperture can be defined in the longitudinal wall 120 such that the working channel can also be at least partially open. Additionally, in another aspect, the elongate body can be substantially planar, as illustrated in FIG. 9. That is, in this aspect, at least a portion of the at least one longitudinal wall 120 of the elongate body can extend in a plane. In yet another aspect, a handle 126 can be coupled to or formed integrally with a portion of a proximal end 140 of the elongate body 100.

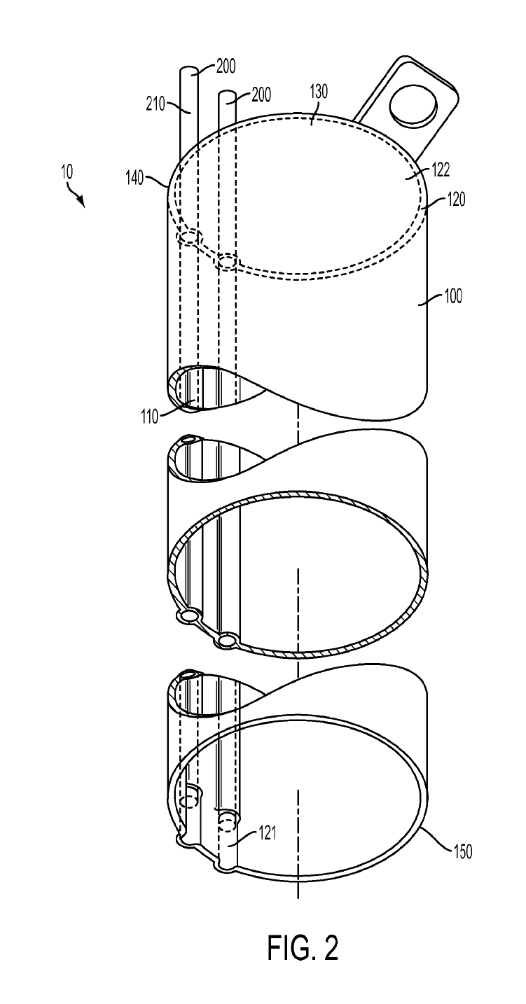

In one aspect, the at least one longitudinal pathway 110 can extend from the proximal end 140 of the elongate body 100 to a distal end 150 of the elongate body. In another aspect, the at least one longitudinal pathway 110 can be defined at least partially within the longitudinal wall 120 of the elongate body 100, as illustrated in FIGS. 2-5. That is, at least a portion of the longitudinal pathway 110 can be defined between an interior face 122 and an exterior face 123 of the longitudinal wall.

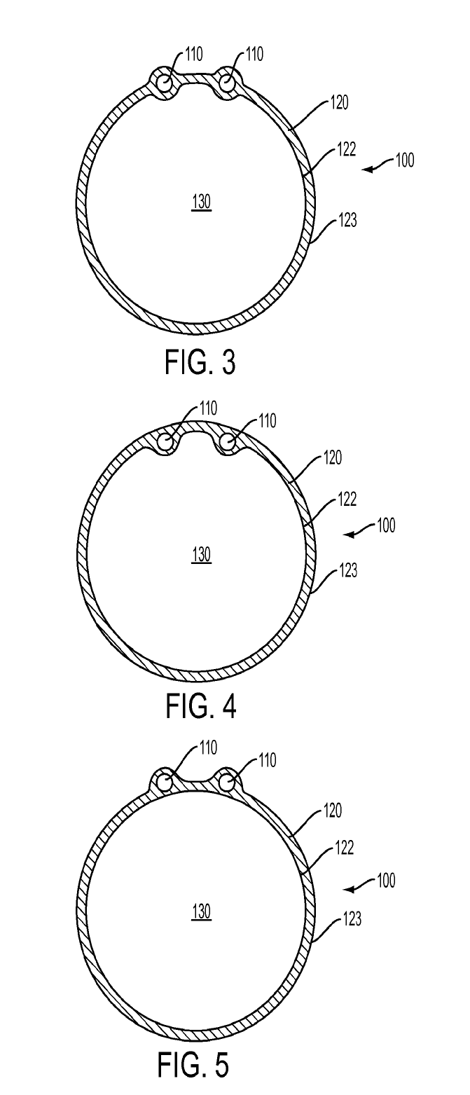

The at least one longitudinal pathway 110 can be separate and spaced from the working channel 130, according to one aspect. In another aspect, the at least one longitudinal pathway can be spaced from the working channel by a portion of the longitudinal wall 120 as illustrated in at least FIGS. 3-5. In yet another aspect, the longitudinal pathway can be completely circumferentially enclosed by the longitudinal wall 120, or optionally, a portion of the longitudinal pathway can be open such that the longitudinal pathway 110 is not completely circumferentially enclosed by the longitudinal wall 120. When the elongate body 100 is substantially tubular and the longitudinal pathway 110 is partially open, regardless of the cross-sectional shape of the elongate body, the partially open portion of the longitudinal pathway 110 can be an opening defined in the interior face 122 of the longitudinal wall 120, according to one aspect. In this aspect, positioning the partially open portion of the longitudinal pathway 110 on the interior face 122 can, for example, help to prevent debris from getting into the longitudinal pathway 110. In another example, positioning the partially open portion of the longitudinal pathway 110 on the interior face 122 can provide a greater field of vision to the surgeon as described more fully below.

The longitudinal wall 120 can be substantially uniform in thickness, or optionally, the longitudinal wall can be thicker in a predetermined area, such as, for example and without limitation, the area of the longitudinal pathway 110, in order to accommodate the longitudinal pathway. In one aspect, if the longitudinal wall 120 is thicker in the area of the longitudinal pathway, the longitudinal wall can protrude substantially evenly inward and outward (as illustrated in FIG. 3), or the longitudinal wall 120 can protrude substantially inward toward the working channel 130 (as illustrated in FIG. 4) or substantially outward (as illustrated in FIG. 5) creating a larger area for the working channel 130.

In one aspect, the working channel 130 can be dimensioned to accommodate the retractor and/or any other surgical instruments 125 that are required for use by the surgeon. For example, and not meant to be limiting, the working channel 130 can measure from about 8 millimeters to about 45 millimeters in diameter. The working channel can also measure from about 10 millimeters to about 40 millimeters in diameter. Additionally, the working channel can measure from about 15 millimeters to about 35 millimeters in diameter.

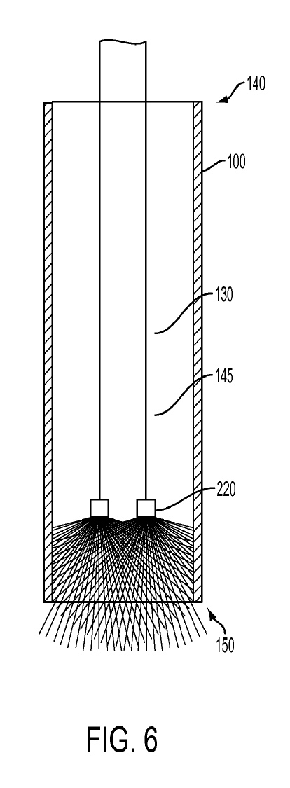

The vision module 200 can enable the surgeon to externally see an interior viewpoint of the patient during surgery. In one aspect, the vision module comprises an optical transmissive element 210 that is configured to convey images such as, a glass rod lens, a camera 220 and the like. In another aspect, at least a portion of the optical transmissive element can be positioned in the longitudinal pathway 110 of the elongate body. In a further aspect, the optical transmissive element 210 can extend from substantially the proximal end 140 of the elongate body 100 to substantially the distal end 150 of the elongate body. In another aspect, the optical transmissive element can extend from substantially the proximal end 140 of the elongate body 100 to a central portion 145 of the elongate body, as illustrated in FIG. 6.

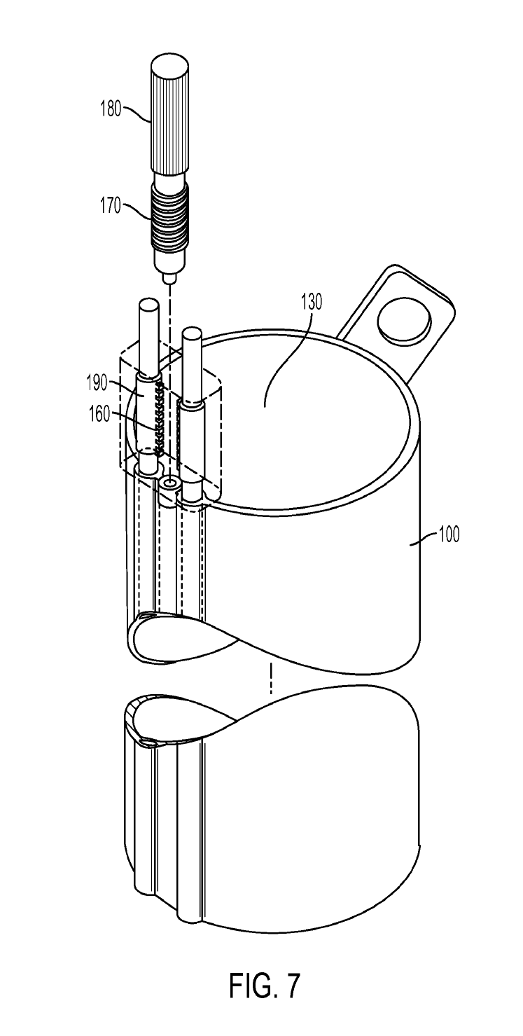

At least a portion of the vision module 200 can be selectively movable to enable changing the field of view. For example, at least a portion of the vision module can be movable in the longitudinal direction. In another example, at least a portion of the vision module 200 can be rotatable about a longitudinal axis L_{P} of the at least one longitudinal pathway 110. In one aspect, and with reference to FIG. 7, the vision module 200 can be operatively connected at or near the proximal end 140 of the elongate body 100 to bands 190 having external threads 160 or similarly functioning gear devices. In this aspect, a worm gear 170 can be positioned on the elongate body 100, which, when turned, can raise or lower the vision module longitudinally. The worm gear can be operatively connected to a knob 180 which can be manually or mechanically actuated. In one aspect, the vision module comprises an eyepiece (not shown) at or near its proximal end. The eyepiece can also be configured to magnify the image for easier viewing by the surgeon. The system can also comprise a light source to illuminate the surgical field.

The optical transmissive element 210 can comprise glass, plastic, fiber optic, or other light carrying material. In one exemplified aspect, the longitudinal pathway 110 can be open toward the distal end 150 of the elongate body 100 to enable viewing of the entire distal portion of the working channel 130 of the elongate body. That is, a viewing window 121 can be defined in a portion of the interior face 122 of the longitudinal wall 120 to enable viewing of the entire distal portion of the working channel 130 from the at least one longitudinal pathway 110 as illustrated in FIG. 2. The amount of the distal portion of the longitudinal pathway 110 needed to accomplish this (that is, the size of the viewing window) depends upon the diameter of the working channel 130. As one skilled in the art can appreciate, the larger the diameter of the working channel, the larger the viewing window 121 at the distal end of the longitudinal pathway needs to be.

In one aspect, the vision module 200 can comprise a plurality of optical transmissive elements 210 to enable multidimensional viewing. In another aspect, the vision module can also comprise one or more mirrors (not shown) to bend the image path and change the field of vision for the surgeon.

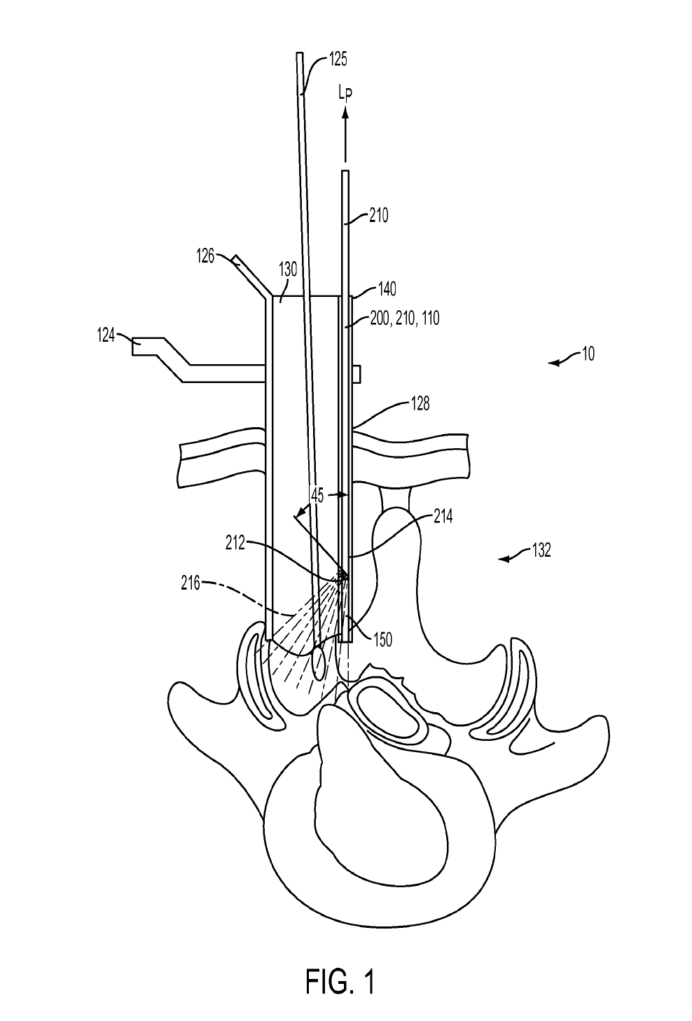

Referring again to FIG. 1, in one aspect, a lens 212 can be positioned at a distal end 214 of the optical transmissive element 210. In this aspect, the lens can be configured to position the field of view 216 in a predetermined direction. In another aspect, the lens 212 can be at an acute angle relative to the longitudinal axis L_{P} of the at least one longitudinal pathway 110. For example, the acute angle can be about 45 degrees (as illustrated). Optionally, in other examples, the acute angle can be between about 15 degrees and 75 degrees, between about 30 degrees and 60 degrees, and between about 40 and 50 degrees. In another aspect, the acute angle between the lens and the longitudinal axis L_{P} can be selectively adjustable by the surgeon. For example, the surgeon can position the lens 212 at first angle to view a first view, and then change the position of the lens to a second angle to view a second view.

In another aspect, the vision module comprises at least one small camera or nano-camera 220 positioned therein the longitudinal pathway 110. The camera feed can be delivered to a screen for the surgeon to view the surgical area.

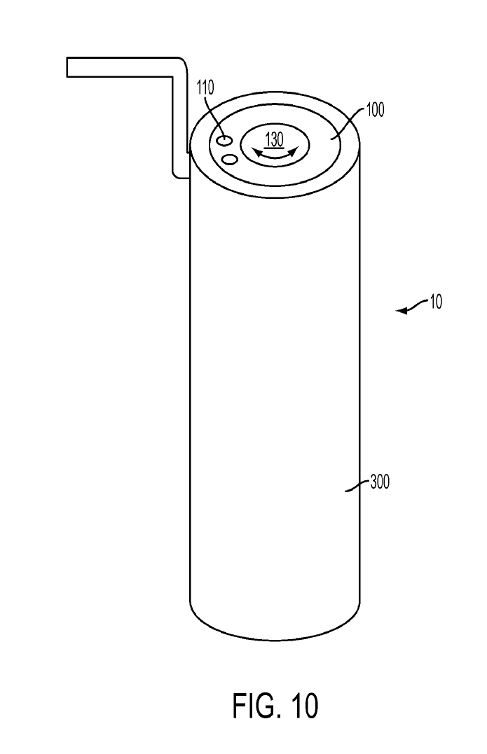

In an exemplified aspect and with reference to FIG. 10, the retractor vision system 10 further comprises an outer retractor tube 300 that substantially circumferentially surrounds the elongate body 100. In this aspect, the elongate body and retractor tube can be shaped complimentarily with one another such that the elongate body can rotate with respect to the outer retractor tube 300. In this way, the elongate body 100 (and the vision module 200), can rotate while the outer retractor tube remains substantially fixed, retracting the surrounding tissue. Thus, this feature permits the vision module 200 to move independently from the outer retractor tube.

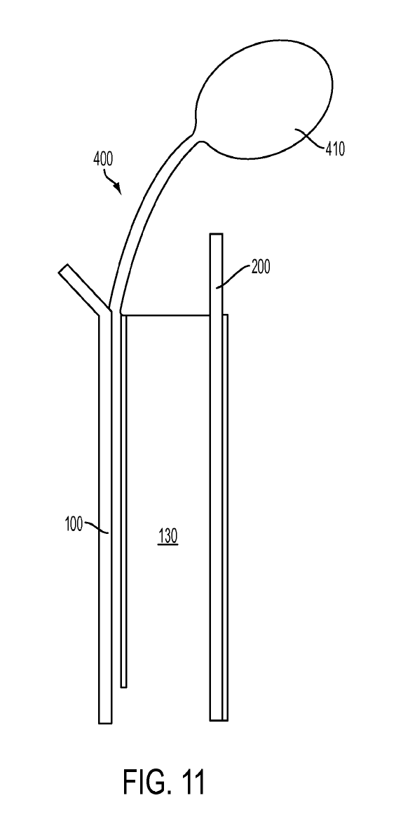

Referring now to FIG. 11, in another aspect, the retractor vision system 10 further comprises an irrigation system 400 to irrigate the surgical area. The irrigation system 400 can comprise an irrigation tube 405 connected to an external bulb 410 to pump fluid into the surgical area. However, other known irrigation systems are also contemplated. The irrigation system can be a separate component or it can be integral with the elongate body 100. The irrigation system 400 can comprise the irrigation tube 405 that is positioned at least partially within a longitudinal pathway 110 in the longitudinal wall 120 of the elongate body.

In still another aspect, the retractor vision system can comprise a suction system 500. The suction system can comprise a suction tube 505 operatively connected to a suction pump 510, wherein the suction tube is positionable within a longitudinal pathway 110 or can be positionable within the working channel 130. The suction tube can have one or more apertures along the wall and/or the distal end of the elongate body 100. In yet another aspect, a longitudinal pathway itself can be the suction tube 505. In this aspect, an external suction tube can be configured to engage the longitudinal pathway with, for example and not meant to be limiting, a luer lock or the like. The longitudinal pathway 110, used as the suction pathway, can have one or more openings along the longitudinal length of the pathway in communication with the working channel and/or at the distal end 150 of the elongate body.

In use, the surgeon can make an incision 128 in the patient 132, and a portion of the elongate body 100 can be inserted through the incision until the distal end 150 of the elongate body is in the desired position relative to the patient. The optical transmissive element 210 can be inserted into the at least one longitudinal pathway 110 of the elongate body until the distal end 214 of the optical transmissive element is in the desired position relative to the patient 132 and/or the elongate body. Optionally, the optical transmissive element 210 can be inserted into the longitudinal pathway of the elongate body prior to insertion of the elongate body through the incision. Images from inside the patient can be transmitted by the optical transmissive element 210 to the surgeon through an eyepiece, a display monitor and the like. In one aspect, the position and/or orientation of the optical transmissive element 210 and/or the elongate body can be adjusted as desired by the surgeon so that the desired field of view 216 is visible. For example, the external threads 160 and worm gear 170 can be used to adjust the longitudinal position of the optical transmissive element. Upon achieving the desired field of view, surgical instruments can be inserted through the working channel 130 of the elongate body 100 and to the desired position on the patient.

Although several aspects of the invention have been disclosed in the foregoing specification, it is understood by those skilled in the art that many modifications and other aspects of the invention will come to mind to which the invention pertains, having the benefit of the teaching presented in the foregoing description and associated drawings. It is thus understood that the invention is not limited to the specific aspects disclosed hereinabove, and that many modifications and other aspects are intended to be included within the scope of the appended claims. Moreover, although specific terms are employed herein, as well as in the claims that follow, they are used only in a generic and descriptive sense, and not for the purposes of limiting the described invention.

What is claimed is:

1. A retractor vision system for a surgeon comprising:
an elongate body defining at least one longitudinal pathway and a separate working channel therein, wherein the working channel is configured to provide access for at least one surgical instrument to a surgical site on a patient, and wherein the at least one longitudinal pathway is defined at least partially within at least one longitudinal wall of the elongate body; and
a vision module sized and shaped to fit at least partially in the at least one longitudinal pathway of the elongate body, wherein the vision module enables the surgeon to externally see an interior viewpoint of the patient during surgery, and wherein the vision module comprises at least one optical transmissive element configured to convey images.

2. The retractor vision system of claim 1, wherein the at least one optical transmissive element comprises a glass rod lens.

3. The retractor vision system of claim 1, wherein the at least one optical transmissive element comprises a camera.

4. The retractor vision system of claim 1, wherein the at least one optical transmissive element comprises a plurality of optical transmissive elements.

5. The retractor vision system of claim 1, wherein the at least one longitudinal pathway extends from a proximal end of the elongate body to a distal end of the elongate body.

6. The retractor vision system of claim 1, wherein the elongate body is tubular and comprises at least one longitudinal wall that defines at least a portion of the working channel.

7. The retractor vision system of claim 1, wherein the at least one longitudinal pathway is completely circumferentially enclosed by the longitudinal wall.

8. The retractor vision system of claim 1, wherein a viewing window is defined in a portion of an interior face of the longitudinal wall to enable viewing of a distal portion of the working channel from the at least one longitudinal pathway by the vision module.

9. The retractor vision system of claim 1, wherein the elongate body is substantially circular in cross-sectional shape.

10. The retractor vision system of claim 1, wherein at least a portion of the vision module is selectively movable relative to the elongate body to enable changing the field of view of the vision module.

11. The retractor vision system of claim 10, wherein at least a portion of the vision module is selectively rotatable about a longitudinal axis of the vision module.

12. The retractor vision system of claim 10, wherein at least a portion of the vision module is selectively movable in a longitudinal direction.

13. The retractor vision system of claim 12, wherein the vision module further comprises external threads configured to operably engage a worm gear positioned in the elongate body, and wherein turning the worm gears moves the vision module longitudinally.

14. The retractor vision system of claim 1, further comprising an outer retractor tube that circumferentially surrounds the elongate body, wherein the elongate body and retractor tube are shaped complimentarily with one another such that the elongate body can rotate with respect to the outer retractor tube.

15. The retractor vision system of claim 1, further comprising an irrigation system to irrigate a surgical area, wherein the irrigation system comprises an irrigation tube operatively connected to an external bulb to pump fluid into the surgical area, and wherein at least a portion of the irrigation tube is positioned within a longitudinal pathway of the elongate body.

16. The retractor vision system of claim 1, further comprising a suction system to provide suction to a surgical area, wherein the suction system comprises a suction tube operatively connected to a suction pump, wherein at least a portion of the suction tube is positioned within a longitudinal pathway of the elongate body.

17. A retractor vision system for a surgeon comprising:
an elongate body having at least one longitudinal wall, wherein the at least one longitudinal wall defines at least one longitudinal pathway therein, wherein the at least one longitudinal wall defines a working channel therein, wherein the at least one longitudinal pathway is defined at least partially within the at least one longitudinal wall, wherein the at least one longitudinal pathway is spaced from the working channel by a portion of the at least one longitudinal wall, and wherein the working channel is configured to provide access for at least one surgical instrument to a surgical site on a patient; and
a vision module sized and shaped to fit at least partially in the at least one longitudinal pathway of the elongate body, wherein the vision module enables the surgeon to externally see an interior viewpoint of the patient during surgery, and wherein the vision module comprises at least one optical transmissive element configured to convey images.

18. The retractor vision system of claim 17, wherein the at least one optical transmissive element comprises a glass rod lens.

19. The retractor vision system of claim 17, wherein the at least one optical transmissive element comprises a camera.

20. The retractor vision system of claim 17, wherein a viewing window is defined in a portion of an interior face of the longitudinal wall to enable viewing of a distal portion of the working channel from the at least one longitudinal pathway by the vision module.
